(19) United States
(12) Patent Application Publication
(10) Pub. No.: US 2011/0245198 A1
(43) Pub. Date: Oct. 6, 2011

(54) ENHANCED TREATMENTS TO KILL OR DEBILITATE PATHOGENIC MICROORGANISMS OF A MAMMALIAN BODY

(76) Inventors: Nikolay Nifantiev, Moscow (RU); Gerhard D. Wieland, Jena (DE)

(21) Appl. No.: 12/727,383

(22) Filed: Mar. 19, 2010

Related U.S. Application Data
(60) Provisional application No. 61/162,516, filed on Mar. 23, 2009.

(51) Int. Cl. A61K 31/737; A61P 31/04; A61M 37/00

(52) U.S. Cl. 514/54; 604/20

(57) ABSTRACT

Present invention relates to a composition of matter and a method that improves treatments to inactivate, kill and debilitate pathogenic microorganisms that infect on or within a mammal body, such as Helicobacter pylori. The composition of matter comprises anti-adhesive polysaccharide molecules to abolish or reduce the adhesion of H. pylori to themselves and to gastric mucin without affecting the viability of either bacteria or gastric epithelial cells. Polysaccharides isolated from seaweed are preferred anti-adhesive materials and fucoidans is a most preferred embodiment. To facilitate bacteria eradication, methods include the administration of fucoidans before, during and/or after other killing and destroying physical or chemical therapy. The inhibition or impairment of the mechanism of bacterial adhesion due to the treatment with fucoidans is aimed to diminish colonization and pathogenesis of pathogenic bacteria by making them more fragile or susceptible to killing or destroying therapies. The combined use of fucoidans with PDT is a preferred method to eradicate H. Pylori in the gastrointestinal tract.

### DOMESTIC PRIORITY UNDER 35 USC 119(e)

This application claims the benefit and priority of U.S. Provisional Application Ser. No. 61/162,516 filed Mar. 23, 2009, entitled “Enhanced Treatments To Kill Or Debilitate Microorganisms Of Human Body” by Nikolay Nifantiev, which is incorporated by reference herein.

### BACKGROUND OF THE INVENTION

1. Field of the Invention

The present invention is related to the treatment of ailments on or within the human body caused by pathogenic microorganisms. More particularly the invention relates to the development of a method and composition of matter to improve therapeutic effectiveness of current treatments for infectious disease on or within patient's body caused by the pathogenic microorganism Helicobacter pylori.

2. Invention Disclosure Statement

There are a number of pathogenic microorganisms that produce human and animal infectious diseases on and within a mammalian body. Microorganisms may infect body tissues such as the skin, or body cavities including, but not limited to the stomach, the mouth, the nasal cavity, the lungs, the bowel, the peritoneal cavity and the urinary tract. An adequate example of this kind of pathogenic microorganism is the gram-negative, spiral shaped, micro-aerophilic bacterium H. pylori, shown a be responsible for infections on various areas of stomach and duodenum.

Although infection with H. pylori can be asymptomatic, in a significant minority of infected people, it is associated with serious conditions including gastritis, gastric ulcer, duodenal ulcer, gastric cancer and gastric lymphoma. Thus, the eradication of the infection may improve symptoms including dyspepsia, gastritis and peptic and duodenal ulcers, and may prevent gastric cancer.

Before recognizing that a wide variety of cases of stomach ailments were due to bacteria, treatments were based on medicines that neutralize gastric acid or decrease its production. After the discovery of bacteria's role in these infectious diseases many antimicrobial therapies to eradicate H. pylori infection have been developed.

Treatment of an H. pylori infection requires a combination of drugs, consisting of one or more antibiotics in combination with an acid-suppressive drug, (proton pump inhibitors [PPIs] or H₂-receptor antagonists) or a bismuth component. The standard first-line therapy is a one week triple therapy consisting of the antibiotics and a proton pump inhibitor. Metronidazole, clarithromycin, amoxicillin, tetracycline, and bismuth are the most widely used drugs for the treatment of pylori. Occasionally, ciprofloxacin, moxifloxacin, levoiloxacin, furazolidone, and rifabutin are used, in particular fbr third-line treatment. Although bismuth-based quadruple therapy, consisting of two antibiotics, a PPI, and a bismuth compound is more effective against H. pylori than triple therapy based on PM and H2-receptor antagonists, the quadruple regimen is usually not prescribed as first-line treatment because of the more complex dosing schedule and side-effects.

Unfortunately, none of the above mentioned antibiotics is effective enough to eliminate H. pylori when given as monotherapy, thus with these conventional treatments there is a need for a combination therapy to effectively eradicate the pathogenic microorganism.

Moreover, owing to the ability of microorganisms to withstand the effects of antibiotics there is limited efficacy of the available therapies because of the development of antimicrobial resistance. This results in initial treatment failure and requires additional rounds of antibiotic therapy. Since resistance against the commonly used antibiotics is increasing, fluoroquinolones (e.g., ciprofloxacin, moxifloxacin, and levafloxacin), nitrofurans (e.g., furazolidone), and rifamycins e.g., rifabutin) are occasionally being used in second and third-line therapies. Initial results obtained with these antibiotics were promising, but antibiotic resistance against these drugs soon emerged.

As the treatment requires the use of at least three concomitant different antibiotics there is potentially a significant number of side effects such as nausea, diarrhea, altered sense of taste and sometimes allergic reactions. Owing to bacteria's abilities, such as formation of biofilms, survival in extremely acidic environment of the stomach and natural selection through random mutation, H. pylori can colonize and establish pathogenesis while developing antibiotic resistance. This combination of harmful abilities is responsible for the extremely difficult eradication of such infectious diseases. As numerous studies have shown that antibiotic resistance substantially impairs the efficacy of anti-H. pylori therapy it would be advantageous to have an antimicrobial therapy that excludes the use of antibiotics, their inherent side-effects and the development of drug resistance.

Alternatively, it has been shown in some cases that H. pylori infection is associated with gastric cancer, developing the disease in any part of the stomach and spreading throughout the stomach and to other organs; particularly the esophagus and the small intestine. Thus, it would be propitious to effectively eradicate the pathogenic microorganism that potentially causes the growth of malignant cells.

A therapy having significant potential advantages over antibiotic therapy for bacterial infection and chemotherapy for cancer treatment is PhotoDynamic Therapy is (PDT). This treatment includes pretreatment with a photosensitizing drug, followed by illumination of the treatment area to kill cells having a high concentration of the drug, which preferentially absorbs light at specific wavelengths. This therapy is usually used to debilitate or destroy malignant tumor cells that have preferentially retained the photosensitizing drug, while preserving adjacent normal tissue. This proves to be an interesting alternative to treat ailments on or within human body as it provides a safe, effective and localized treatment. Nonetheless, careful considerations have to be taken into account related to the characteristics of the photosensitizing drug used as well as the appropriate exciting light source, as the effectiveness of the PDT treatment mainly relies on these considerations. In an attempt to treat these infectious diseases, U.S. Pat. No. 6,890,346 B2 by Ganz et al, disclosed a surgical apparatus and method for treating ailments in body cavities of a patient. The treatment involves the use of light radiation for debilitating or killing microorganisms without serious destruction of body tissue. Nonetheless this therapy is yet not sufficiently effective as radiation fails to completely kill or debilitate bacteria, owing to the protective biofilm formed while bacteria adhered into mucus layers.

Aiming to provide higher light output power and more physical flexibility of the probes Friedman et al. designed a flexible array disclosed in the U.S. Pat. No. 7,135,034 B2. Although the radiation delivery to the interior surface of body cavities was modified, the treatment continues to be ineffective to treat the ailment within body cavities as the way that radiation is delivered is not sufficient to remove the protective biofilm and kill or debilitate all pathogen bacteria.

A chemiluminescent light source emitting visible light for biotherapy is disclosed by Tolkoff et al. in U.S. Pat. No. 7,255,691 B2, intending to utilize naturally-occurring chemicals produced by human body and endogenously photosensitive chemicals produced by light-sensitive bacteria, such as H. pylori. The main advantage of this source of radiation provides a wavelength capable of exciting endogenous bacterial porphyrins which in turn release free radicals damaging the pathogen bacteria. Conversely, the disclosure proposes techniques for altering pH levels, increasing the temperature of the stomach or applying iodine solutions as the chemiluminescent light source fails to address and to effectively modify the local environment in the stomach to facilitate the eradication by light by making the bacteria more fragile or susceptible.

In another attempt for treating an ailment in a body cavity of a patient, apparatuses and methods are disclosed by Levin et al. in International Publication N° WO 2008/066943 A2. The apparatus is inserted into a body cavity, a balloon is inflated around an array of optical fibers ends and is the irradiated to kill or debilitate microorganisms. The method may also comprise the use of two fluids, one containing optical modifiers and other containing adjuvants to enhance light therapy.

Once again, intending to have a more effective therapy by improving probes flexibility and illumination methods, Friedman et al. in U.S. Pat. No. 7,261,730 B2 disclosed a device to provide high light output power for performing therapeutic medical procedures in body lumens. Even though the light output power is higher, is still less than fully effective due to the bacteria's ability to adhere and penetrate the mucus layer while forming a protective biofilm.

Thus, a great need exists for an effective treatment of ailments on or within a human body aimed to debilitate or kill pathogenic microorganisms responsible for infectious diseases. There also exists a need for a therapy focused on destruction, damage or debilitation of the protective biofilm formed by bacteria in order to facilitate their eradication by making the bacteria more fragile or susceptible.

### OBJECTIVES AND BRIEF SUMMARY OF THE INVENTION

The aim of this invention is to provide a method and a composition of matter to enhance the inactivating, debilitating and killing effect, by physical or chemical means of energy, of microorganism causing infectious diseases on or within a mammal body.

It is another objective to provide a method and a composition of matter to treat pathogenic microorganisms, such as H. pylori, causing infectious diseases on or within the gastrointestinal tract, facilitating bacteria eradication by physical or chemical means of energy by making them more fragile or susceptible.

Yet, another objective is to provide a composition of matter to abolish or reduce the adhesion of H. pylori to themselves in order to build a biofilm or to gastric mucin to diminish the colonization and establishment of the pathogenesis that bacteria produce by adhering to the gastrointestinal mucus surface.

A further objective is to provide a composition of matter based on polysaccharides that may prevent H. pylori from binding to gastric mucin optimally at acidic pH environments, without affecting the viability of either bacteria or gastric epithelial cells, thus favoring its anti-adhesive action in a gastric environment.

It is still another objective to use a class of fucoidans as a treatment administered before, during and/or after other treatments, to avoid the adhesion of H. pylori to gastric mucin, thus impeding the adhesion of bacteria onto gastrointestinal tract.

Briefly stated the present invention relates to a composition of matter and a method that improves treatments to inactivate, kill and debilitate pathogenic microorganisms that infect on or within a mammalian body, such as Helicobacter pylori. The composition of matter comprises anti-adhesive polysaccharide molecules to abolish or reduce the adhesion of H. pylori to themselves and to gastric mucin without affecting the viability of either bacteria or gastric epithelial cells. Polysaccharides isolated from seaweed are preferred anti-adhesive materials and fucoidans is a most preferred embodiment. To facilitate bacteria eradication, methods include the administration of fucoidans before, during and/or after other killing, inactivating and destroying physical or chemical therapies. The inhibition or impairment of the mechanism of bacterial adhesion due to the treatment with fucoidans is aimed to diminish colonization and pathogenesis of pathogenic bacteria by making them more fragile or susceptible to killing or destroying therapies. The combined use of fucoidans with PDT is a preferred method to eradicate H. Pylori in the gastrointestinal tract.

The above and other objects, features and advantages of the present invention will become apparent from the following description read in conjunction with the accompanying drawings, (in which like reference numbers in different drawings to designate the same elements.)

### BRIEF DESCRIPTION OF FIGURES

FIGS. 1a and b show one preferred embodiment of the invention in which the chemical formula of homofucose backbone chains in brown seaweed fucoidans are depicted.

FIG. 2, in another embodiment of the invention, exemplifies structural elements of fucoidan's homofucose backbone chains.

### DETAILED DESCRIPTION OF PREFERRED EMBODIMENTS

The present invention is further illustrated by the example of treatment of H. pylori infections within a mammal's gastrointestinal tract, but it should be understood that the invention is not limited thereby.

Present invention intends to obstruct the adhesion of H. pylori to gastric mucin without affecting the viability of either bacteria or gastric epithelial cells, as an improvement for treating ailments in the gastrointestinal tract. Due to the harsh environment caused by the gastric fluids, a mammal's gastrointestinal tract is covered with a highly impermeable polymer matrix of a mucus layer that consists of gastric mucin, which compromises high-molecular-mass glycoproteins. H. pylori takes advantage of this protection by inhabiting in the mucus lining and once there, protects itself from the stomach acid by secreting an enzyme which converts urea from saliva and gastric juices into bicarbonate and ammonia and neutralize the acids around it. In order for H. pylori to colonize the underlying gastric epithelium the bacteria must be able to adhere to and penetrate the mucus layer. Thus, the present invention provides an advantageous method and composition of matter to avoid or diminish H. pylori infection by effectively obstructing the adhesion of H. pylori to themselves and to gastric mucin in order to facilitate the eradication of H. pylori by associated physical or chemical means. Since adhesion to gastric mucin is essentially the initial stage in H. pylori colonization and establishment of pathogenesis, this is the first objective to accomplish.

In a preferred embodiment, present invention provides a composition of matter comprising naturally occurring polysaccharides as a potential anti-adhesive agent against H. pylori colonization of gastric mucin. The reason for this is that certain polysaccharides, such as fucoidans, bind to carbohydrate-binding proteins (lectins) presented on the surface of H. pylori, which have shown to possess mucin-binding activity. Anti-adhesive properties of fucoidans may not allow colonization and pathogenesis of H. pylori by impeding bacterial adhesion onto gastrointestinal mucus surface and bacterial adhesion between themselves in order to build a biofilm by blocking special sugar epitopes on the bacterial cell wall. In such an anti-adhesive-based therapy, it is important to obstruct H. pylori adhesion to gastric mucin, as the impermeable nature of the mucus gel barrier may mean that high-molecular-weight polysaccharide compounds cannot cross the gel layer to block binding sites present on the surface of underlying cells.

Fucoidans represent a class of fucose-enriched sulfated polysaccharide found in the extracellular matrix of various species of brown seaweed and in some marine invertebrates and have proven to successfully act as anti-adhesives on the binding of H. pylori to gastric mucin. Polysaccharides isolated from seaweed are preferred anti-adhesive materials and fucoidans is a most preferred embodiment.

Depending on their origin, fucoidans may have different fucose or sulfate content and diverse structural features of their polysaccharide backbones. This in turn will characterize their biological activities, including but not limited to anti-inflammatory, antiangiogenic, anticoagulant, and anti-adhesive actions.

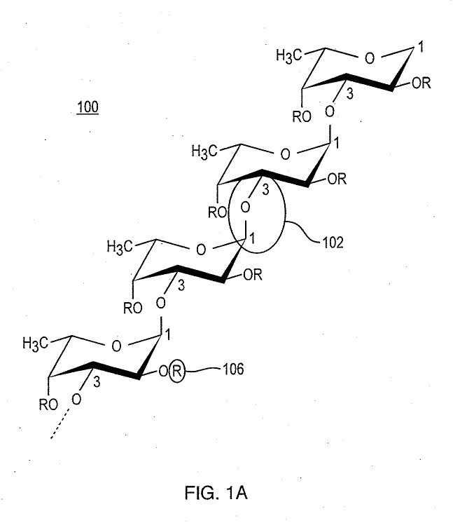

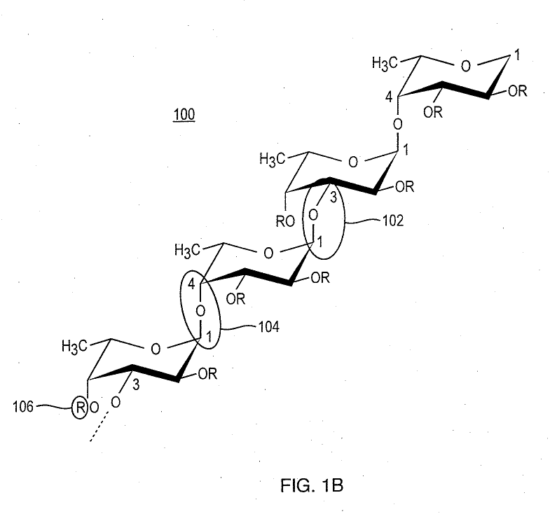

Seaweed fucoidans represent mixtures of structurally related polysaccharides with certain variations in their content of carbohydrate units and non-carbohydrate substituent. Due to their heterogeneity, another preferred embodiment of polysaccharide-based composition of matter refers to brown seaweed fucoidans with a structure similar to those depicted in FIGS. 1a and b, where the chemical formula of two types of homofucose backbone chains 100 is shown. One type of homofucose backbone chains is constructed of repeating (1→3) 102 linked α-L-fucopyranose residues whereas the other form contains alternating (→3) 102 and (1→4) 104 linked α-L-fucopyranose residues. In both cases R 106 depicts the places of potential attachment of carbohydrate and non-carbohydrate substituent.

Still in another preferred embodiment, carbohydrate substituent may be α-L-facopyranose (Fuc), α-D-glucuronic acid (GlcA), galactose (Gal), mannose (Man), Xylose (Xyl) and glucose (Glc); and non-carbohydrate substituent may be sulfate and acetyl groups.

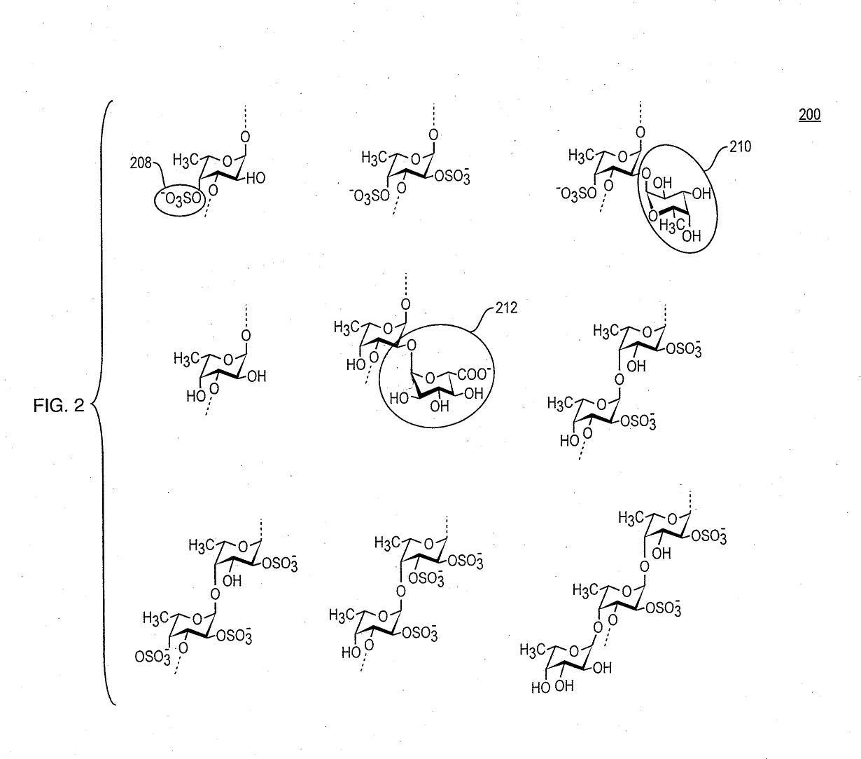

The homofucose backbone chains of fucoidans may have, in another preferred embodiment, different structural elements as depicted in FIG. 2. The structural elements 200 may have a sulfate group 208 as residue 106; a α-L-fucopyranosyl 210 residue and/or a α-L-glucuronyl residue.

In another embodiment, methods to improve alternative treatments for ailments on or within a mammal body and to facilitate bacteria eradication include the administration of present invention polysaccharide-based composition of matter in combination with an energy source such as irradiation by light or other physical or chemical means. In another embodiment, the methods to facilitate bacteria eradication on or within a mammalian body include the administration of fucoidans before, during and/or after other killing and destroying physical or chemical therapies. The anti-adhesion property of fucoidan may facilitate bacteria eradication by making them more fragile or susceptible to additional inactivating, killing and destroying therapies. A method to eradicate H. pylori from body cavity of a mammal comprises the steps of 1) administering an anti-adhering therapeutic compound; 2) introducing a chemical or physical means of energy; and 3) eliminating said pathogenic microbes. Preferably, anti-adhering therapeutic compound is present invention's polysaccharide-based composition of matter, i.e. fucoidan. Improvement over other therapies is attained by the administration of fucoidans, before, during and/or after other treatments aimed to kill, inactivate or destroy pathogenic microorganisms.

In a preferred embodiment, a method for H. Pylori eradication in the gastrointestinal tract is to combine the use of fucoidans with PDT, including if necessary, administration of an external photosensitizing agent followed by illumination of the area to be treated. The method comprises the steps of 1) administering an anti-adhering therapeutic compound; 2) where appropriate administrating a suitable photosensitizer; 3) irradiating free floating microbes in body cavity; and 4) eliminating microbes by photo-destruction.

Irradiation is performed with the aid of an electromagnetic radiation source preferably emitting radiation of wavelength between about 375 and 475 nm. However, preferably emitting radiation wavelength matches an absorption peak of photosensitive species in said body cavity and/or an absorption peak of an external photosensitizing agent administered, as appropriate. One or more radiation wavelengths may be delivered simultaneously or one after the other, in an intercalating manner. Electromagnetic radiation source includes coherent and incoherent radiation sources such as laser radiation source, light emitting diodes source, lamp radiation source (incandescent, xenon arc and metal halide lamps) and/or others known in the art. In one embodiment, the electromagnetic radiation source is a laser source and emits laser radiation. Depending on the medical application electromagnetic radiation may be delivered from inside a body cavity or lumen or interstitially by optical fibers with or without diffuser tips. Preferable external photosensitizing agent administered to enhance PDT effect is Safranin O, nevertheless other photosensitizing agent may also be used.

In another embodiment, a method for treating or preventing gastric cancer and lymphoma caused by H. Pylori infection, combines the use of fucoidans with photosensitizing agents and PDT, including drugs used to impair cell division or to induce apoptosis. This method of treatment eradicates H. pylori and allows the treatment or prevention of gastric cancer and lymphoma caused by the pathogenic microorganism.

The present invention is further illustrated by the following example, but is not limited thereby.

### Example 1

Present example shows the inactivation of H. pylori by Antibacterial Photodynamic Therapy (APDT) as described in this invention. Briefly, H. pylori bacteria were pregrown in Brain-Heart-Infusion-MHO-medium at 37° C. under microaerophilic atmospheric condition for one week. These bacterial cultures have been diluted to an optical density of 0.100 at 600 nm (OD-600 nm). 200 μl aliquots of these bacterial suspensions were placed in a deep well micro titer plates. The APDT was conducted using coherent light of a 405 nm blue/violet diode laser system equipped with a Medlight-Fiber with microlense diffuser. The diffuser was placed 2 mm over the surface of the bacterial suspension and the illumination times were 5, and 30 minutes in the experiments without FUCOIDAN and 5, 10 and 15 or 30 minutes in the experimental set with FUCOIDAN-primed bacteria. Every single experiment was conducted in triplicate. After illumination, the treated samples and the mock controls were used as an inocolumn for 10 ml of fresh BHI medium. 48 hours after the treatment, the OD-600 nm of the sample was used to assess the efficacy of the treatment. The results showed that by using the light of a 405 nm laser system it was possible to inactivate about 92-98% of H. pylori using endogen Coproporphyrins in the bacterial cell wall as photosensitizers. Table 1 summarizes the illumination time and the results obtained after 48 hours of solely illumination of bacteria by using laser light at 405 nm.

TABLE 1
Inactivation of H. pylori by APDT only using laser light at 405 nm.

| | Illumination | OD600nm | OD600nm | Efficacy- |
|---|---|---|---|---|
| Exper- | at 405 nm | growth control | 48 h after | Inactive bacteria |
| iment | [min] | 48 h without light | illumination | [%] |
| 1 | 5′ | 0.594 | 0.037 | 93.74 |
| | 10′ | 0.611 | 0.021 | 96.56 |
| | 30′ | 0.599 | 0.021 | 96.49 |
| 2 | 5′ | 0.587 | 0.015 | 97.44 |
| | 10′ | 0.633 | 0.04 | 93.67 |
| | 30′ | 0.599 | 0.034 | 94.31 |
| 3 | 5′ | 0.887 | 0.028 | 96.84 |
| | 10′ | 0.901 | 0.012 | 98.67 |
| | 30′ | 0.874 | 0.074 | 91.52 |
| 4 | 5′ | 1.243 | 0.033 | 97.34 |
| | 10′ | 0.888 | 0.019 | 97.86 |
| | 30′ | 0.977 | 0.017 | 98.26 |
| 5 | 5′ | 0.857 | 0.033 | 96.14 |
| | 10′ | 0.984 | 0.038 | 96.13 |
| | 30′ | 0.896 | 0.028 | 96.87 |
| 6 | 5′ | 0.874 | 0.037 | 95.76 |
| | 10′ | 0.853 | 0.037 | 95.66 |
| | 30′ | 0.963 | 0.033 | 96.57 |
| 7 | 5′ | 0.587 | 0.04 | 93.17 |
| | 10′ | 0.633 | 0.05 | 92.09 |
| | 30′ | 0.588 | 0.039 | 93.36 |

In a second experimental set, we aimed for a higher inactivation percentage because for the medical use of a APDT-assay it is relevant to reach inactivation rates higher than 99%. FUCOIDAN is known to hinder bacterial adhesion. So, H. pylori is no more able to produce Biofilms or bind to endothelial cells. To test the effect of FUCOIDAN in this combined approach to inactivate H. pylori the bacteria again were pregrown in Brain-Heart-Infusion-(BHI)-medium at 37° C. under microaerophilic atmospheric condition for 1 week. FUCOIDAN was added to the growth medium in concentration ranges from 0.5 to 500 μg/ml. The experimental set was conducted using FUCOIDAN in a concentration of 20 μg/ml. These bacterial cultures differ from FUCOIDAN-free cultures microscopically as you cannot find any grape-like aggregates. Only planctonic single bacteria can be detected. These cultures were diluted to an optical density of 0.100 at 600 nm. 200 μl aliquots of these bacterial suspensions were placed in a deep well micro titer plates. After the following illumination with the 405 nm laser system and subsequent growing of treated and mock control cultures, the OD-600 nm was measured. Table 2 shows the results of this second set of experiments adding FUCOIDAN. So, by using FUCOIDAN to hinder bacterial adhesion we were able to inactivate more than 99.5 percent of H. pylori. These data clearly demonstrate the efficacy of the combined approach—pretreatment with FUCOIDAN and subsequent APDT- to eliminate Helicobacter pylori.

TABLE 2
Inactivation of fucoidan-primed H. pylori by APDT

| | | OD600nm | | Efficacy- |
|---|---|---|---|---|
| | Illumination | growth | OD600nm | Inactive |
| | at 405 nm | control 48 h | 48 h after | bacteria |
| Experiment | [min] | without light | illumination | [%] |
| 1 | 5′ | 0.795 | 0.031 | 96.10 |
| with Fucoidan | 10′ | 0.798 | 0.021 | 97.37 |
| | 30′ | 0.886 | 0.004 | 99.55 |
| 2 | 5′ | 0.971 | 0.008 | 99.18 |
| with Fucoidan | 10′ | 0.974 | 0.031 | 96.81 |
| | 30′ | 0.985 | 0.024 | 97.56 |
| 3 | 1′ | 0.961 | 0.023 | 97.60 |
| with Fucoidan | 5′ | 0.974 | 0.012 | 98.77 |
| | 15′ | 0.985 | 0.006 | 99.39 |
| 4 | 1′ | 0.961 | 0.017 | 98.23 |
| with Fucoidan | 5′ | 0.974 | 0.012 | 98.77 |
| | 15′ | 0.993 | 0.004 | 99.60 |
| 5 | 1′ | 0.961 | 0.012 | 98.75 |
| with Fucoidan | 5′ | 0.934 | 0.017 | 98.18 |
| | 15′ | 0.997 | 0.005 | 99.50 |
| 6 | 1′ | 0.971 | 0.017 | 98.25 |
| with Fucoidan | 5′ | 0.984 | 0.009 | 99.08 |
| | 15′ | 0.927 | 0.004 | 99.57 |
| 7 | 1′ | 0.961 | 0.017 | 98.23 |
| with Fucoidan | 5′ | 0.949 | 0.011 | 98.84 |
| | 15′ | 0.921 | 0.003 | 99.67 |

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A polysaccharide-based composition of matter which is used in combination with an energy source, selected from the group consisting of irradiation by light or other chemical or physical means, to enhance killing and debilitating pathogenic microorganisms that infect on/within a mammal body.

2. The polysaccharide-based composition of matter, according to claim 1, which is used in combination with PhotoDynamic Therapy (PDT) to enhance killing or debilitating microorganisms that infect on or within the mammal body.

3. The polysaccharide-based composition of matter, according to claim 1, wherein said polysaccharide is a naturally occurring polysaccharide.

4. The polysaccharide-based composition of matter according to claim 1, wherein said polysaccharide is fucoidans.

5. The polysaccharide-based composition of matter according to claim 1, wherein said polysaccharide is extracted from seaweeds.

6. The polysaccharide-based composition of matter, according to claim 1, wherein a target is said pathogenic microbe is H. pylori, a Gram-negative bacterium colonizing mucosal lining of gastrointestinal tract of humans.

7. An improved treatment method for killing, debilitating and eliminating pathogenic microbes from body cavity of animal mammal comprising of the steps of:
administering an anti-adhering therapeutic compound;
where appropriate administrating a suitable photosensitizer;
irradiating free floating microbes in body cavity; and
eliminating microbes by photo-destruction.

8. The improved treatment method according to claim 7, wherein said step of irradiating free microbes comprises applying of light energy, whose wavelength matches at least one absorption peak of a photosensitive species in said body cavity or at least one absorption peak of an external photosensitizing agent administered, as appropriate.

9. An improved treatment method for killing, debilitating and eliminating pathogenic microbes from body cavity of a mammal comprising of the steps of: administering an anti-adhering therapeutic compound; introducing a chemical or physical means of energy; and eliminating said pathogenic microbes.

10. The improved treatment method according to claim 7, wherein said step of administering an anti-adhering therapeutic compound is performed before, during and/or after irradiating free floating microbes in body cavity to kill, debilitate or eliminate microbes.

11. The improved treatment method according to claim 9, wherein said step of administering an anti-adhering therapeutic compound is performed before, during and/or after using other chemical or physical means to kill, debilitate or eliminate microbes.

12. The improved treatment method according to claim 7, wherein said pathogenic microbe is H. pylori, a Gram-negative bacterium colonizing mucosal lining of gastrointestinal tract of humans.

13. The improved treatment method according to claim 7, wherein said step of irradiating free floating microbes in body cavity is done with an electromagnetic radiation source selected from the group of; laser radiation sources, light emitting diode sources, lamp radiation sources and combinations of them.

14. The improved treatment method according to claim 13, wherein said electromagnetic radiation source operates at least at one radiation wavelength between about 375 and 475 nm.

15. The improved treatment method according to claim 13, wherein said radiation wavelength is delivered in a manner selected from the group of simultaneously with another radiation wavelength and one after the other, in an intercalating manner.

16. The improved treatment method according to claim 13, wherein said electromagnetic radiation source is a laser source and emits laser radiation.

17. The improved treatment method according to claim 13, wherein said laser radiation is delivered from inside a body cavity or lumen or interstitially by optical fibers.

18. The improved treatment method according to claim 7, wherein said anti-adhering therapeutic agent is a naturally occurring polysaccharide.

19. The improved treatment method according to claim 18, wherein said polysaccharide is extracted from seaweeds.

20. The improved treatment method according to claim 19 wherein said polysaccharide is preferably fucoidans.

21. The improved treatment method according to claim 9 wherein said anti-adhering therapeutic agent is a naturally occurring polysaccharide.

22. The improved treatment method according to claim 21, wherein said polysaccharide is extracted from seaweeds.

23. The improved treatment method according to claim 22 wherein said polysaccharide is preferably fucoidans.

24. The improved treatment method according to claim 9, wherein said pathogenic microbe is H. pylori, a Gram-negative bacterium colonizing mucosal lining of gastrointestinal tract of humans.
